(19) United States
(12) Patent Application Publication
(10) Pub. No.: US 2013/0202535 A1
(43) Pub. Date: Aug. 8, 2013

(54) X-RAY VISIBLE MEDICAL DEVICE AND PREPARATION METHOD THEREOF

(71) Applicant: SNU R&DB Foundation, Seoul (KR)

(72) Inventors: Tae Hyun CHOI, Seoul (KR); Young Bin CHOY, Gyeonggi-do (KR); Seok Min KWON, Seoul (KR); Catherine Ann SHASTEEN, Seoul (KR); Sung Yoon CHOI, Gyeonggi-do (KR); Min PARK, Seoul (KR); Suk Wha KIM, Seoul (KR)

(73) Assignee: SNU R&DB FOUNDATION, Seoul (KR)

(21) Appl. No.: 13/621,744

(22) Filed: Sep. 17, 2012

(30) Foreign Application Priority Data
Feb. 2, 2012 (KR) 10-2012-0010991

(51) Int. Cl. A61B 17/68; A61K 49/04

(52) U.S. Cl. CPC A61B 17/68; A61K 49/04
USPC 424/9.4; 156/242; 156/60; 427/2.1; 606/76

(57) ABSTRACT

The present invention relates to an X-ray visible medical device comprising a medical device and a layer bound onto the medical device, wherein the layer is composed of an X-ray contrast material or a composite of the X-ray contrast material and a biocompatible polymer. Also disclosed is a method for preparing the X-ray visible medical device.

### BACKGROUND OF THE INVENTION

1. Field of the Invention

The present invention relates to an X-ray visible medical device and a preparation method thereof, and more particularly to an X-ray visible medical device, which has X-ray contrast properties while substantially maintaining the mechanical property and biocompatibility thereof, and a preparation method thereof.

2. Description of the Prior Art

A variety of bone fractures are treated using internal fixation devices that provide suitable support and mechanical fixation for bone treatment. Such fixation devices were made of metals, typically a titanium alloy, a cobalt-chromium alloy and stainless steel. In some cases of bone fixation, for example, in the case in which the fixation device (e.g., metallic fixation devices) excessively protects bone fracture sites to interfere with the growth of bone, the fixation device should be removed later. In this case, removal of the fixture device is carried out after the bone has been completely cured.

A fixation plate made of a biodegradable polymer is an effective means for fixation of bone fractures, which eliminates the need for removal surgery. The biodegradable polymer is degraded slowly by hydrolysis or enzymatic degradation in vivo to generate products, which are harmless to the body and metabolized by cells or removed from the body. In this way, this fixation plate does not require additional removal surgery and can be significantly comfortable and convenient after bone fixation.

The biodegradable polymer should have sufficient strength such that the fixation device can resist continuous stress for the time required for bone density growth. Currently, biodegradable polymer-based fixation devices are being clinically used, and these devices were found to be most suitable in areas which do not support body weight. Polymer-based devices are widely distributed over the entire list of fixation devices, including plates, screws and pins. Thus, the entire fixation system can be made completely of biodegradable components, and due to their high biocompatibility, the polymer-based fixation devices are highly promising in the fields of orthopedic surgery and plastic surgery.

However, these biodegradable polymer-based fixation devices, which are essential medical devices in orthopedic surgery or plastic surgery, are not visible to general X-ray-based imaging systems which are used to assess bone fixation. Thus, it is difficult to examine the position of the fixation device in vivo after surgery and to confirm suitable fixation during the treatment procedure. The suitable selection of the position of the fixation device is most important in the initial stage of treatment in order to treat bone properly. If bone is improperly treated, additional surgery can be required for correction. It is beneficial to analyze bone treatment and the positions of fixation devices using X-ray radiation during the initial several weeks of treatment.

Accordingly, the present inventor has conducted studies in view of the above-described facts and prepared an X-ray visible medical device comprising a medical device and a layer bound onto the medical device, wherein the layer is composed of an X-ray contrast material or a composite of the X-ray contrast material and a biocompatible polymer. Also, the present inventor has examined the X-ray contrast effect of the prepared medical device over a specific period of time after in vivo implantation of the medical device, and as a result, has found that the medical device is permanently visible to X-rays or is visible to X-rays for a specific period of time and can be biodegraded with time due to the degradation of the bound layer, thereby completing the present invention.

### SUMMARY OF THE INVENTION

It is an object of the present invention to provide an X-ray visible medical device comprising: a medical device; and a layer bound onto the medical device, wherein the layer is composed of an X-ray contrast material or a composite of the X-ray contrast material and a biocompatible polymer.

Another object of the present invention is to provide a method for preparing the X-ray visible medical device.

### DETAILED DESCRIPTION OF INVENTION

To achieve the above objects, the present invention provides an X-ray visible medical device comprising: a medical device; and a layer bound onto the medical device, wherein the layer is composed of an X-ray contrast material or a composite of the X-ray contrast material and a biocompatible polymer.

In the present invention, the medical device may be any one selected from the group consisting of a plate, a screw or a pin, which is for bone fixation or bone fracture treatment, but is not limited thereto.

In the present invention, the medical device may be made of a biocompatible polymer. Preferably, the medical device may be made of a biodegradable polymer, but is not limited thereto.

As used herein, the term “biocompatible polymer” refers to a biocompatible polymer that causes almost no rejection reaction when being implanted in vivo. Specifically, the medical device of the present invention may be any polymer material which is biocompatible and can be used for bone fixation or bone fracture treatment. More preferably, the medical device is made of a biodegradable polymer, which can be biodegraded after in vivo implantation and eliminates the need for additional removal surgery. Herein, the biodegradable polymer is preferably a polymer which can be biodegraded over 1-36 months in vivo. If the biodegradable polymer is degradable within a period shorter than the lower limit of the above range, it will be biodegraded within the bone fracture treatment period, making it difficult to maintain the bond fixation effect, and if it cannot be degraded in a period longer than the upper limit, it can cause inflammatory reactions in vivo. Specifically, examples of the biodegradable polymer include, but are not limited to, poly(lactic acid), poly(glycolic acid), poly(lactic-co-glycolic acid), poly(ethylene glycol), poly(trimethylene carbonate), poly(caprolactone), poly(dioxanone) and the like. In addition, examples of polymers, which are biocompatible but not biodegradable, include, but are not limited to, poly(methylmethacrylate), polyethylene (PE), polytetrafluoroethylene (PTFE), polyvinyl chloride (PVC), polydimethylsiloxane (PDMS), polyurethane (PU) and the like. The biocompatible polymer may be a biodegradable polymer, a non-biodegradable polymer, or a copolymer thereof, or a blend of two or more polymers. Specifically, the biocompatible polymer may be one or more selected from the group consisting of poly(lactic acid), poly(glycolic acid), poly(lactic-co-glycolic acid), poly(ethylene glycol), poly(trimethylene carbonate), poly(caprolactone), poly(dioxanone), poly(methylmethacrylate), polyethylene (PE), polytetrafluoroethylene (PTFE), polyvinyl chloride (PVC), polydimethylsiloxane (PDMS), polyurethane (PU), and copolymers thereof, but is not limited thereto.

The X-ray visible medical device according to the present invention is prepared such that it is visible to X-rays, without reducing its mechanical strength. Specifically, it is prepared by making a layer composed of either an X-ray contrast material or a composite of an X-ray contrast material and a biocompatible polymer and binding the layer to the surface of a medical device or a groove formed on the surface.

Alternatively, it is prepared by binding a layer, composed of an X-ray contrast material or a composite of the X-ray contrast and a biocompatible polymer, directly to the surface of a medical device or a groove formed on the surface. The layer may be in the form of a film, particles or a pattern.

In the present invention, in the case in which a layer composed of an X-ray contrast material or a composite of the X-ray contrast material and a biocompatible polymer is manufactured separately, a method such as solution casting or melt casting may be used. In addition, if the layer composed of the X-ray contrast material or the composite of the X-ray contrast material and the biocompatible polymer is bound directly to the medical device, a method may be used, such as spreading, spraying, dropping, dipping, coating, dabbing with sponge, stamping, rolling, brushing, spray casting, or electrospinning.

In the present invention, the biocompatible polymer that may be used in the layer is a biocompatible polymer which causes almost no rejection reaction after being implanted in vivo, as described above with respect to the material of the medical device. It is any polymer which is biocompatible and can be used for bone fixation or bone fracture treatment. Among biocompatible polymers, a biodegradable polymer is preferably used which does not eliminate the need for additional removal surgery after implantation in vivo. The biodegradation period of the biodegradable polymer that may be used in the layer may have biodegredable period equal, shorter or longer than the biodegradable period of the biodegradable polymer used in the medical device. Preferably, the biodegradation period of the biodegradable polymer that may be used in the layer is shorter than the biodegradable period of the biodegradable polymer used in the medical device. Specifically, the biodegradable polymer that may be used in the layer may be a polymer which can be biodegraded over 2 weeks to 6 months in vivo. If the biodegradable polymer can be biodegraded within a period shorter than the lower limit of the above range, the X-ray visible period can be shortened, and if it cannot be biodegraded in a period longer than the upper limit, it can cause inflammatory reactions in vivo. Specifically, examples of the biodegradable polymer include, but are not limited to, poly(lactic acid), poly(glycolic acid), poly(lactic-co-glycolic acid), poly(ethylene glycol), poly(trimethylene carbonate), poly(caprolactone), poly(dioxanone) and the like. In addition, examples of polymers, which are biocompatible but not biodegradable, include, but are not limited to, poly(methylmethacrylate), polyethylene (PE), polytetrafluoroethylene (PTFE), polyvinyl chloride (PVC), polydimethylsiloxane (PDMS), polyurethane (PU) and the like. The biocompatible polymer may be a biodegradable polymer, a non-biodegradable polymer, or a copolymer thereof, or a blend of two or more polymers. Specifically, the biocompatible polymer may be one or more selected from the group consisting of poly(lactic acid), poly(glycolic acid), poly(lactic-co-glycolic acid), poly(ethylene glycol), poly(trimethylene carbonate), poly(caprolactone), poly(dioxanone), poly(methylmethacrylate), polyethylene (PE), polytetrafluoroethylene (PTFE), polyvinyl chloride (PVC), polydimethylsiloxane (PDMS), polyurethane (PU), and copolymers thereof, but is not limited thereto.

In the present invention, the X-ray contrast material may be one or more selected from the group consisting of calcium phosphate, barium sulfate, potassium iodide, metals such as gold or iron, and combinations thereof, but is not limited thereto. Preferably, beta-tricalcium phosphate (β-TCP) may be used.

In the present invention, the X-ray contrast material or the composite of the X-ray contrast material and the biocompatible polymer may be patterned and bound onto the medical device. The shape of the pattern may be selected from among various shapes, including a circle, a square, a triangle, a polygon, a straight line, a curved line, a dot, letters, or combinations thereof, but is not limited thereto.

In the present invention, the layer may be bound onto the medical device by an adhesive. Specifically, the adhesive may be one or more selected from among: one or more organic solvents selected from the group consisting of dimethylformamide (DMF), tetrahydrofuran (THF) and methylchloroform (MC); a polymer solution of one or more polymers selected from the group consisting of poly(methyl methacrylate), poly(lactic acid), poly(glycolic acid), poly(lactic-co-glycolic acid), poly(ethylene glycol), poly(trimethylene carbonate), and copolymers thereof, in the one or more organic solvents; and one or more biocompatible adhesives selected from the group consisting of fibrin glue, polysaccharides and mucopolysaccharide; but is not limited thereto.

The present invention also provides a method for preparing an X-ray visible medical device, comprising the steps of:

(1) preparing a film composed of an X-ray contrast material and a biocompatible polymer; and

(2) binding the film onto a medical device.

Step (1) consists of preparing a film composed of an X-ray contrast material and a biocompatible polymer by mixing the two materials thereof.

Specifically, step (1) may comprise the following steps:
(1-1) mixing a biocompatible polymer solution or melt with the X-ray contrast material;

(1-2) placing the mixture in a mold to prepare a film; and

(1-3) drying the film.

Step (1-1) is a step of mixing the biocompatible polymer solution or melt with the X-ray contrast material. In this step, the biocompatible polymer solution or melt is mixed with the X-ray contrast material to prepare the mixture for preparing a film.

The biocompatible polymer solution or melt is obtained by dissolving a biocompatible polymer in a solvent. The kind of biocompatible polymer is as described above with respect to the medical device, and the solvent may be selected depending on the kind of biocompatible polymer. Specifically, the solvent may be an organic solvent, such as dimethylformamide (DMF), tetrahydrofuran (THF) or methylchloroform (MC), but is not limited thereto.

The biocompatible polymer melt is obtained by melting a biocompatible polymer without using a separate solvent.

The kind of X-ray contrast material is as described above with respect to the medical device.

The mixture obtained by mixing the biocompatible polymer solution or melt with the X-ray contrast material may be in the form of a liquid or dough suitable of being molded into a film.

Step (1-2) is a step of placing the mixture in a mold to prepare a film. In this step, the mixture is molded into a film.

The mold can be manufactured using a master mold having the same shape as that of the film to be formed. The master mold may be manufactured to have a desired shape using a poly(methyl methacrylate) (PMMA) sheet, a poly(carbonate) (PC) sheet, a poly(ethylene terephthalate) (PET) sheet, a poly(ethylene naphthalate) (PEN) sheet or the like. When the mold is manufactured using the master mold, poly(dimethylsiloxane) (PDMS) may, for example, be used as the material of the mold. In addition, any conventional material used in the art may be used as the material of the mold.

In addition, the shape of the film may be selected from among various shapes, including a circle, a square, a triangle or a polygon, but is not limited thereto.

Step (1-3) is a step of drying the film. In this step, the film is dried to be cured.

A method that can be used for the drying is not specifically limited. However, in order to protect the properties of the material, freeze drying is preferably used. Particularly, in order to remove a residual solvent in a vacuum, vacuum freeze drying is preferably used. The freeze drying is preferably carried out at a temperature ranging from −40° C. to −50° C. Also, the drying may be carried out for 12-48 hours.

Step (2) is a step of binding the film onto a medical device. In this step, the film comprising the X-ray contrast material is bound onto the medical device such that the medical device is visible to X-rays.

Specifically, step (2) may comprise the following steps:

(2-1) coating the medical device with an adhesive;

(2-2) placing the film composed of the X-ray contrast material and the biocompatible polymer on the medical device coated with the adhesive; and

(2-3) drying the medical device having the film placed thereon.

Step (2-1) is a step of coating the medical device with the adhesive. In this step, the adhesive is coated on the medical device such that the film is easily bound to the medical device.

The kind of adhesive that may be used in step (2-1) is as described above with respect to the medical device. Meanwhile, coating with the adhesive can be carried out using a conventional method such as spreading, spraying, dropping, brushing, dipping, or the like.

Step (2-2) is a step of placing the film composed of the composite of the X-ray contrast material and the biocompatible polymer on the medical device coated with the adhesive. In this step, the film is placed on the desired position of the medical device coated with the adhesive to bind the film.

Step (2-3) is a step of drying the medical film having the film placed thereon. In this step, the medical device having the film placed thereon is dried to bind the film to the medical device.

A method that can be used for the drying is not specifically limited. However, in order to protect the properties of the material, freeze drying is preferably used. Particularly, in order to remove a residual solvent in a high vacuum, vacuum freeze drying is preferably used. The freeze drying is preferably carried out at a temperature ranging from −40° C. to −50° C. Also, the drying may be carried out for 12-48 hours.

The present invention also provides a method for preparing a X-ray visible medical device, comprising the steps of:

(1) preparing an X-ray contrast material or a mixture of the X-ray contrast material and a biocompatible polymer;

(2) coating a medical device with an adhesive; and

(3) binding the X-ray contrast material or mixture of step 1) onto the adhesive-coated medical device of step (2) to form a layer on the medical device.

Preferably, the method may further comprise, after step (3), step (4) of drying the medical device having the layer bound thereto.

Step (1) is a step of preparing a mixture of an X-ray contrast material or a mixture of the X-ray contrast material or a biocompatible polymer. In this step, the X-ray contrast material or the X-ray contrast material/biocompatible polymer mixture, which is to be used to form a layer, is prepared.

The X-ray contrast material may be in a form of powder suitable for forming a layer.

The biocompatible polymer may be mixed as powder, a solution in a solvent, or a melt obtained without using any solvent. Specifically, the X-ray contrast material/biocompatible polymer mixture may be used as dough, powder or liquid, which is suitable for forming a layer.

Herein, the kind of biocompatible polymer and the kind of X-ray contrast material are as described above with respect to the medical device. The solvent may be suitably selected depending on the kind of biocompatible polymer. Specifically, the solvent may be an organic solvent such as dimethylformamide (DMF), tetrahydrofuran (THF) or methylchloroform (MC), but is not limited thereto.

Step (2) is a step of coating the medical device with an adhesive. In this step, the medical device is pre-treated with the adhesive for binding the X-ray contrast material or the X-ray contrast material/biocompatible polymer mixture.

The kind of adhesive which can be used herein is as described above with respect to the medical device. Meanwhile, coating with the adhesive may be carried out using a conventional method such as spreading, spraying, dropping, brushing, dipping, or the like.

Step (3) is a step of binding the X-ray contrast material or mixture of step (1) onto the adhesive-coated medical device to form a layer on the medical device. In this step, the X-ray contrast material or the X-ray contrast material/biocompatible polymer mixture is bound onto the adhesive-coated medical device to form a layer on the medical device.

The binding in step (3) may be carried out using spreading, spraying, dropping, dipping, coating, dabbing with sponge, stamping, rolling, brushing, spray casting or electrospinning, but is not limited to.

In addition, the binding in step (3) may be carried out using a mask to pattern the layer.

Herein, the shape of the pattern may be selected from various shapes, including a circle, a square, a triangle, a polygon, a straight line, a curved line, a dot, letters, or combinations thereof, but is not limited thereto.

Particularly, if the layer is patterned into letters or patterned into a combination of various figures, lines or dots such that the upper and lower sides thereof are distinguished from each other, the upper and lower sides of the medical device can be distinguished from each other when they are irradiated with X-rays.

Step (4) is a step of drying the medical device having the layer formed thereon. In this step, the medical device having the layer formed thereon is dried in order to completely bind the layer to the medical device.

A method that can be used for the drying is not specifically limited. However, in order to protect the properties of the material, freeze drying is preferably used. Particularly, in order to remove a residual solvent in a high vacuum, vacuum freeze drying is preferably used. The freeze drying is preferably carried out at a temperature ranging from −40° C. to −50° C. Also, the drying may be carried out for 12-48 hours.

The present invention also provides a method for preparing an X-ray visible medical device, comprising the steps of:

(1) preparing a mixture of an X-ray contrast material and an adhesive or a mixture of the X-ray contrast material, a biocompatible polymer and a biocompatible polymer; and

(2) binding the mixture onto a medical device to form a layer on the medical device.

Preferably, the method may further comprise, after step (2), step (3) of drying the medical device having the layer formed thereon.

Step (1) is a step of preparing a mixture of an X-ray contrast material and an adhesive or a mixture of the X-ray contrast material, a biocompatible polymer and an adhesive. In this step, either the X-ray contrast material or the X-ray contrast material and the biocompatible polymer are mixed with the adhesive for binding it in order to prepare a mixture for forming a layer.

The X-ray contrast material/adhesive mixture may be used as dough or liquid, which is suitable for forming a layer. The state of the X-ray contrast material/adhesive mixture can vary depending on the state of the adhesive used.

The biocompatible polymer may be mixed as a powder, a solution in a solvent, or a melt obtained without using any solvent. The X-ray contrast material/biocompatible polymer/adhesive mixture may be used as a dough or a liquid, which is suitable for forming a layer. The state of the X-ray contrast material/biocompatible polymer/adhesive mixture can vary depending on the property of the biocompatible polymer and the adhesive.

The kinds of biocompatible polymer, X-ray contrast material and adhesive which can be used in this method, are as described above with respect to the medical device. In addition, the solvent can be suitably selected depending on the kind of biocompatible polymer. Specifically, the solvent may be an organic solvent such as dimethylformamide (DMF), tetrahydrofuran (THF) or methylchloroform (MC), but is not limited thereto.

Step (2) is a step of binding the mixture onto a medical device. In this step, the mixture containing the X-ray contrast material is bound directly onto the medical device to form a layer which is visible to X-rays.

The binding step (2) may be carried out using spreading, spraying, dropping, dipping, coating, dabbing with sponge, stamping, rolling, brushing, spray casting or electrospinning, but is not limited thereto.

In addition, the binding in step (2) may be carried out using a mask to pattern the layer.

Herein, the shape of the pattern may be selected from various shapes, including a circle, a square, a triangle, a polygon, a straight line, a curved line, a dot, letters, or combinations thereof, but is not limited thereto.

Particularly, if the layer is patterned into letters or patterned into a combination of various figures, lines and dots such that the upper and lower sides thereof are distinguished from each other, the upper and lower sides of the medical device can be distinguished from each other when they are irradiated with X-rays.

Step (3) is a step of drying the medical device having the layer formed thereon. In this step, the medical device having the layer formed thereon is dried in order to completely bind the layer to the medical device.

A method that can be used for the drying is not specifically limited. However, in order to protect the properties of the material, freeze drying is preferably used. Particularly, in order to remove a residual solvent in a high vacuum, vacuum freeze drying is preferably used. The freeze drying is preferably carried out at a temperature ranging from −40° C. to −50° C. Also, the drying may be carried out for 12-48 hours.

The present invention also provides a method for preparing an X-ray visible medical device, comprising the steps of:

(1) forming a groove on a medical device;

(2) preparing an X-ray contrast material or a mixture of the X-ray contrast and a biocompatible polymer;

(3) packing the X-ray contrast material or X-ray contrast material/biocompatible polymer mixture of step (2) into the groove formed on the medical device in step (1); and to (4) applying an adhesive to the packed groove.

Preferably, the method may further comprise, after step (4), step (5) of drying the additive-applied medical device.

Step (1) is a step of forming a groove on a medical device. In this step, the groove for forming a layer is formed on the medical device.

The groove may be patterned, whereby a layer formed by packing the X-ray contrast material into the groove can also be patterned.

Herein, the shape of the pattern may be selected from various shapes, including a circle, a square, a triangle, a polygon, a straight line, a curved line, a dot, letters, or combinations thereof, but is not limited thereto.

Particularly, if the layer is patterned into letters or patterned into a combination of various figures, lines and dots such that the upper and lower sides thereof are distinguished from each other, the upper and lower sides of the medical device can be distinguished from each other when they are irradiated with X-rays.

Step (2) is a step of preparing an X-ray contrast material or a mixture of the X-ray contrast material and a biocompatible polymer. In this step, the X-ray contrast material or the X-ray contrast material/biocompatible polymer, which is to be used to form a layer, is prepared.

The X-ray contrast material may be used in a powder form suitable for forming a layer.

The biocompatible polymer may be mixed as powder, a solution in a solvent, or a melt obtained without using any solvent. Specifically, the X-ray contrast material/biocompatible polymer mixture may be used as dough, powder or liquid, which are suitable for forming a layer.

Herein, the kind of biocompatible polymer and the kind of X-ray contrast material are as described above with respect to the medical device. The solvent may be suitably selected depending on the kind of biocompatible polymer. Specifically, the solvent may be an organic solvent such as dimethylformamide (DMF), tetrahydrofuran (THF) or methylchloroform (MC), but is not limited thereto.

Step (3) is a step of packing the X-ray contrast material or X-ray contrast material/biocompatible polymer mixture of step (2) into the groove formed on the medical device in step (1). In this step, the X-ray contrast material or the X-ray contrast material/biocompatible polymer mixture is packed into the groove on the medical device to form a layer on the medical device.

Step (4) is a step of applying an adhesive to the packed groove. In this step, the adhesive for binding the X-ray contrast material or the X-ray contrast material/biocompatible polymer mixture is applied to the groove on the medical device.

The kind of adhesive which can be used herein is as described above with respect to the medical device. Meanwhile, application of the adhesive can be carried out using a conventional method such as spreading, spraying, dropping, brushing, dipping, or the like.

Step (5) is a step of drying the medical device to which the adhesive has been applied. In this step, the medical device to which the adhesive has been applied is dried to completely bind the layer, formed in the groove, to the medical device.

A method that can be used for the drying is not specifically limited. However, in order to protect the properties of the material, freeze drying is preferably used. Particularly, in order to remove a residual solvent in a high vacuum, vacuum freeze drying is preferably used. The freeze drying is preferably carried out at a temperature ranging from −40° C. to −50° C. Also, the drying may be carried out for 12-48 hours.

Hereinafter, the present invention will be described in further detail with reference to the accompanying drawings.

The present invention comprises introducing an X-ray contrast layer to the surface of a fixation device so as to enable the fixation device to be analyzed by X-ray irradiation. Introduction of the X-ray contrast layer enables the fixation device to be visible to X-rays while substantially maintaining the strength and biocompatibility thereof. The layer comprises an X-ray contrast material as an essential component, and optionally comprises a biocompatible polymer or an adhesive. Preferably, the biocompatible polymer is a degradable polymer, which is highly biocompatible and was approved for use in implantable devices in vivo.

In fact, the X-ray contrast material takes shape by a biocompatible polymer or an adhesive and is bound to a fixation device. Examples of the X-ray contrast material include β-tricalcium phosphate, a biocompatible, biodegradable osteoconductive ceramic material which is frequently used as orthopedic implant materials, including artificial joints and bone cement coatings. Other examples of the X-ray contrast material include calcium phosphate, barium sulfate, metals, and combinations thereof.

A biodegradable polymer component has a connection with the breakdown of the X-ray contrast layer. The X-ray contrast layer is not a shaped material, but is a material of physically bound particles. As the polymer component breaks down, the X-ray contrast layer also scatters rapidly. The breakdown of the X-ray contrast layer proceeds significantly faster than the degradation of the fixation device. The X-ray contrast layer can break down over a period ranging from 2 weeks to 6 months. However, a fixation device such as a fixation plate requires a significantly long period of time to be degraded. The life expectancy of the X-ray contrast layer can coincide with the period of inflammation following surgery, and after alleviation of the inflammation and breakdown of the X-ray contrast layer, the slim-profile fixation device will remain during the remaining treatment period and the degradation period of the fixation device. This rapid degradation of the X-ray contrast layer prevents inflammation from continuing over a long period of time due to a foreign body reaction.

When a highly biocompatible material is used, X-ray contrast layer particles released in vivo can be naturally metabolized or removed. The two-component system consisting of the X-ray contrast material and the biocompatible polymer, which constitute the X-ray contrast layer, enables various designs and bonding of various materials. First, the X-ray contrast material can be modified to change the X-ray contrast and/or the biocompatibility. Then, the polymer component can be modified to control the breakdown rate of the X-ray contrast layer so as to control the period during which it is visible to X-rays.

FIG. 1 is a schematic view showing a method according to one embodiment of the present invention, wherein an X-ray contrast layer is prepared as a film-type layer and then bound onto a medical device.

As shown in FIG. 1, an X-ray visible medical device according to the present invention can be prepared by preparing an X-ray visible TCP film and attaching the prepared film to a fixation plate.

In one example of the present invention, a PLGA solution was prepared by dissolving the biodegradable polymer PLGA in DMF and mixed with β-TCP powder as an X-ray contrast material, thereby preparing a liquid mixture. The mixture was poured into a mold and freeze-dried. In this way, a film-type TCP coating layer containing β-TCP was obtained by a solution casting method. Then, an adhesive obtained by dissolving PLGA in DMF was applied to a fixation plate, and then the β-TCP-containing coating layer was placed on the adhesive layer, followed by freeze-drying, thereby preparing an X-ray visible fixation plate comprising the β-TCP attached to the fixation plate.

FIG. 2 is a set of optical images showing the appearances of the fixation plate before attachment of the β-TOP layer (a) and after attachment of the β-TCP layer (b).

FIG. 3 shows the results of scanning electron spectroscopy (SEM) of the surface of the (β-TCP layer on the X-ray visible fixation plate prepared as described above. As can be seen in FIG. 3, the TCP particles on the surface of the β-TCP layer were bound densely by PLGA.

FIG. 4 shows X-ray diffraction (XRD) patterns of β-TCP powder and the β-TCP layer attached to the fixation plate. As can be seen in FIG. 4, the β-TCP layer of the present invention shows a crystalline structure, like β-TCP powder.

FIG. 5 shows the results obtained by preparing fixation plates having layer thicknesses of 1.3 mm (a) and 0.5 mm (b), dipping these plates in PBS (pH 7.4), and then subjecting the plates to X-ray imaging at various points of time. As can be seen in FIG. 5, the two kinds of plates were all visible to X-rays for 25 days. Particularly, the plate having a layer thickness of 1.3 mm was highly visible to X-rays.

FIG. 6 shows the results of densitometry conducted to quantify the X-ray imaging results. As can be seen in FIG. 6, X-ray visibility decreased with time in both the plates having layer thicknesses of 1.3 mm (a) and 0.5 mm (b). Particularly, the plate having a layer thickness of 1.3 mm showed higher X-ray visibility, not only at an initial time point, but also over all time points.

FIG. 7 shows the results of in vivo X-ray imaging obtained by surgically fixing the above-prepared plate having a layer thickness of 1.3 mm to a rabbit and then examining a change in X-ray visibility as a function of time. As can be seen in FIG. 7, the X-ray visibility of the plate decreased with time.

FIG. 8 shows the degradation rates of the plates, obtained by quantifying the results of the in vivo X-ray imaging analysis as a function of time. As can be seen in FIG. 8, X-ray visibility decreased with time in both the plates having layer thicknesses of 1.3 mm and 0.5 mm, and decreased rapidly at about 20 days.

FIG. 9 is a schematic view showing a method according to another embodiment of the present invention, wherein a mixture of an X-ray contrast material and a biocompatible polymer is prepared and bound onto, an adhesive-applied medical device by spraying, brushing, dropping or dipping to form a layer on the medical device.

As shown in FIG. 9, an X-ray visible medical device can be prepared by applying an adhesive (binder) to a fixation plate, and then binding a mixture of a biocompatible polymer and an X-ray contrast material to the fixation plate by a method such as spraying, brushing, dropping or dipping to form a layer. Herein, the dipping method can make it easy to take out the dipped fixation plate fixed on a holder.

FIG. 10 is a schematic view showing a method according to still another embodiment of the present invention, wherein a mixture of an X-ray contrast material, a biocompatible material and an adhesive is prepared and bound onto a medical device by a method such as spraying or brushing to form a layer on the medical device.

As shown in FIG. 10, an X-ray visible medical device according to the current embodiment can be prepared by preparing a mixture of an X-ray contrast material and a biocompatible material, and binding the mixture onto a fixation plate by a method such as spraying or brushing to form a layer.

In the binding process, a mask may or may not be. When the mask is not used, the mixture may be bound uniformly to the plate to form a layer covering the entire upper surface of the plate. Meanwhile, when the mask is used, a specific pattern can be formed on the plate according the pattern of the mask used. As shown in FIG. 10, masks having various shapes may be used, and thus the layer can be patterned in a desired shape, such as a lattice, mosaic, linear, circular, triangular, square or polygonal shape.

FIGS. 11 and 12 show examples of possible patterns other than the patterns shown in FIG. 10. As can be seen in FIGS. 11 and 12, all distinguishable shapes, including figures, lines and dots, are possible. In addition, patterns shapes, including figures, lines, dots and letters, which make it possible to distinguish orientation and specific locations, are also possible. Particularly, patterns making it possible to orientation and a specific location make it possible to distinguish the upper and lower sides of a plate.

FIG. 13 is a schematic view showing a method according to still another embodiment of the present invention, wherein a specific amount of an adhesive is absorbed into sponge, and then the sponge is soaked in a mixture of an X-ray contrast material and a biocompatible polymer and pressed against a medical device to form a layer on the medical device.

As shown in FIG. 13, an X-ray visible medical device according to the present invention can be prepared by placing an adhesive in a glass dish, dipping the sponge in the dish so as to sufficiently absorb the adhesive into the sponge, dipping the adhesive-absorbed surface of the sponge in a mixture of an X-ray contrast material and a biodegradable material, and then dabbing the sponge on a fixation device.

FIG. 14 is a schematic view showing a method according to still another embodiment of the present invention, wherein an X-ray contrast material and a biocompatible material are mixed with an adhesive to prepare a mixture, and a specific amount of the mixture is applied to a stamp or a roller, which is then pressed against a medical device to form a layer.

As shown in FIG. 14, an X-ray visible medical device according to the present invention can be prepared by mixing an X-ray contrast material and a biocompatible at a specific ratio, applying the mixture uniformly to a stamp or a roller in a container, and pressing the stamp or roller against a fixation plate to form a layer made of the mixture, followed by freeze drying. Herein, the container is preferably made of a material, PDMS, stainless steel or glass, which have chemical resistance to an organic solvent, because they can store the mixture for a specific time or longer. Moreover, the surface of the stamp or the roller is preferably made of a porous, chemical-resistant, easy-to-process material which can adsorb and hold the mixture. Specifically, the surface of the stamp or the roller may be made of a material such as poly(ethylene), poly(propylene), poly(urethane), silicone rubber or poly(vinyl alcohol), but is not limited thereto.

In addition, in the binding process for forming the layer, a mask may or not be used. When the mask is not used, the mixture may be bound uniformly to the plate to form a layer covering the entire upper surface of the plate. Meanwhile, when the mask is used, a specific pattern can be formed on the plate according the pattern of the mask used. Possible pattern shapes are as described above with respect to FIGS. 10 to 12.

FIG. 15 is a schematic view showing a method according to still another embodiment of the present invention, wherein a powdery X-ray contrast material is prepared, and a medical device is coated with an adhesive, after which the X-ray contrast material is bound to the adhesive-applied medical device to form a layer on the medical device.

As shown in FIG. 15, an X-ray visible medical device according to the present invention can be prepared by continuously blowing the inside of a closed container containing a powdery X-ray contrast material so as to uniformly disperse the contrast material in air, and exposing an adhesive (binder)-applied plate to contrast material for a specific time so as to coat the surface of the plate with contrast particles.

FIG. 16 is a schematic view showing a method according to still another embodiment of the present invention, wherein a groove is formed on a medical device, an X-ray contrast material is packed into the groove, and then an adhesive is applied to the packed groove.

As shown in FIG. 16, an X-ray visible medical device according to the present invention can be prepared by forming a groove on the surface of an absorbable plate, packing an X-ray contrast material in the form of powder, liquid or dough into the groove, spraying, dropping or brushing an adhesive (binder) of a polymer solution onto the surface of the packed groove to fix the X-ray contrast material to the surface, and freeze-drying the resulting structure to remove the solvent. Herein, the groove can be formed by laser machining or according to the shape of a mold in plate injection. Also, the groove can be formed in a desired pattern using a conventional patterning tool. Possible shapes of specific patterns are shown in FIG. 16. In addition, all shapes, including figures, lines and dots, are possible. Further, pattern shapes, including figures, lines, dots and letters, which make it possible to distinguish orientation and a specific location, are also possible. Particularly, patterns making it possible to distinguish the upper and lower sides of a plate.

### EFFECT OF THE INVENTION

As described above, the present invention provides an X-ray visible medical device comprising a medical device and a layer bound onto the medical device, wherein the layer is composed of an X-ray contrast material or a composite of the X-ray contrast material and a biocompatible material. Thus, the present invention can provide an X-ray visible medical device which has X-ray contrast properties while substantially maintaining the mechanical strength and biocompatibility thereof.

### BRIEF DESCRIPTION OF THE DRAWINGS

FIG. 1 is a schematic view showing a process of making a film-type X-ray visible TCP layer and a process of attaching the layer to a fixation plate.

FIG. 2 shows optical images of a fixation plate before attachment of the film-type TCP layer (a) and a fixation plate after attachment of the film-type TCP layer (b).

FIG. 3 shows Scanning Electron Microscope (SEM) images of the TCP layer. (a): 500× magnification; and (b): 2000× magnification.

FIG. 4 shows X-ray diffraction (XRD) patterns of β-TCP powder (a) and a TCP layer sample (b).

FIG. 5 shows the results of in vitro X-ray imaging as a function of time for plates to which each of TCP layers having a thickness of 1.3 mm (a) and 0.5 mm (b) was attached.

FIG. 6 shows the results of in vitro densitometry of plates to which each of TCP layers having a thickness of 1.3 mm (a) and 0.5 mm (b) was attached.

FIG. 7 shows the results of in vivo X-ray imaging in rabbits as a function of time for a plate to which a TCP layer having a thickness of 1.3 mm was attached.

FIG. 8 shows the results of in vivo densitometry in rabbits for plates to which each of TCP layers having a thickness of 1.3 mm (a) and 0.5 mm (b) was attached.

FIG. 9 schematically shows a method wherein a mixture of an X-ray contrast material and a biocompatible polymer is prepared and bound onto an adhesive-applied medical device by a method such as spraying, brushing, dropping or dipping to form a layer on the medical device.

FIG. 10 schematically shows a method wherein a mixture of an X-ray contrast material, a biocompatible polymer and an adhesive is prepared and bound onto a medical device by a method such as spraying or brushing to form a layer on the medical layer.

FIGS. 11 and 12 show possible pattern shapes.

FIG. 13 schematically shows a method wherein a specific amount of an adhesive is absorbed into sponge, after which a mixture of an X-ray contrast material and a biocompatible polymer is applied to the sponge which is then dabbed uniformly against a medical device to form a layer made of the mixture.

FIG. 14 schematically shows a method wherein an adhesive is mixed with an X-ray contrast material and a biodegradable polymer and the mixture is applied to a stamp or a roller, which is then pressed against a medical device to form a layer.

FIG. 15 schematically shows a method wherein a powdery X-ray contrast material is prepared, and a medical article is coated with an adhesive, after which the powdery X-ray contrast material is bound to the adhesive-coated medical device to form a layer.

FIG. 16 schematically shows a method wherein a groove is formed on a medical device, and an X-ray contrast material is packed into the groove, after which an adhesive is applied to the packed groove, thereby binding the X-ray contrast material to the groove to form a layer.

### DETAILED DESCRIPTION OF THE INVENTION

Hereinafter, the present invention will be described in further detail with reference to examples. It is to be understood, however, that these examples are for illustrative purposes only and are not intended to limit the scope of the present invention.

### EXAMPLE 1

### Material and Method

In the present invention, sintered power of β-tricalcium phosphate (β-TCP, Sigma-Aldrich, USA) was used as an X-ray contrast component. The powder was used without further processing, and it was mixed with a polymer solution obtained by dissolving 5050 poly(D,L-lactide-co-glycolide) (Lakeshore Biomaterials, USA) having a molecular weight of about 4.1 kDa in dimethylformamide (DMF). Master molds were prepared using poly(methylmethacrylate) (PMMA) having thicknesses of 1.3 mm and 0.5 mm, and to achieve the desired shape and thickness, coating molds were prepared using poly(dimethylsiloxane) (PDMS) (Slygard 184 Kit, Dow Corning, USA). Injection-molded, 1.5 mm CPS 7×7 mesh plates made of a copolymer of L-lactide, D-lactide and trimethylene carbonate, and 1.5-mm screws, were purchased from Inion (Finland). For in vitro analysis, phosphate buffered saline (PBS, pH 7.4) was purchased from the Biomedical Research Institute, Seoul National University Hospital, and for in vivo analysis, rabbits were purchased.

### EXAMPLE 2

### Preparation of X-ray Contrast Layer and Devices Comprising It

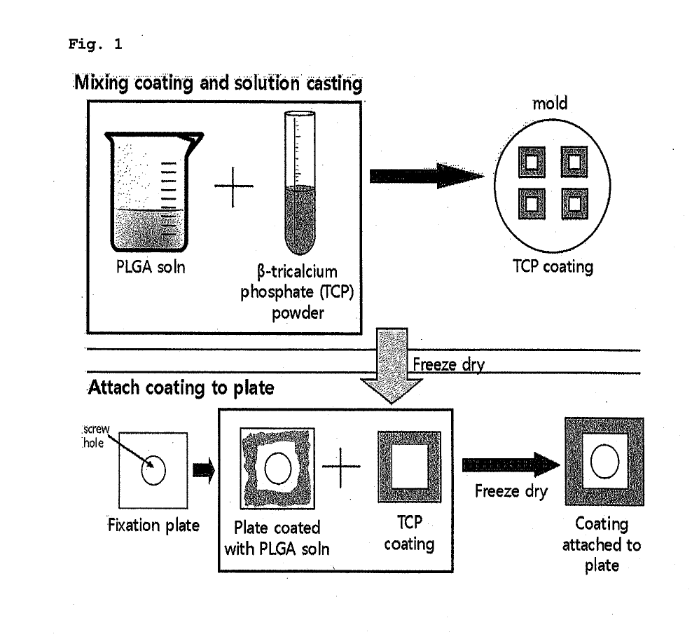

As shown in FIG. 1, an X-ray visible TCP film was prepared and attached to a fixation plate.

PMMAs were cut with a CO₂ laser to prepare master molds in a desired shape. The master molds were in the form of a square frame having a concentric rectangular hole (6.5 mm×2.5 mm). The square frames of the molds had a thickness of 1.3 mm and 0.5 mm, respectively, according to the initial thicknesses of the PMMA.

Using such master molds, an elastomer base was cured using a catalyst according to the manufacturer's instruction, thereby preparing 4 square PDMS molds. The 4 master molds were fixed to a Petri dish. After the PDMS had been completely cured, the molds were taken out from the Petri dish, and the outer surfaces, square openings and rear surfaces of the molds were trimmed to make the surfaces smooth.

β-TCP powder was processed using mortar & pestle in order to reduce the particle size and increase the surface area. A solution of 25% (w/v) PLGA in DMF was added to and mixed with the processed β-TCP powder at a ratio of 200 μL of PLGA solution per 600 mg of β-TCP, so that the weight ratio of β-TCP/PLGA reached 12/1. The prepared β-TCP-PLGA dough was immediately filled in the PDMS molds. An excess of the dough was removed from the molds using a doctor blade, and the filled molds were freeze-dried in a vacuum for 24 hours, thereby preparing TCP films.

The Inion fixation plate was cut to small rectangular pieces having sides of 6.5 mm and comprising a screw hole at the center. In order to attach the dried TCP film to the fixation plate, about 1.5 μL of a solution of 30% (w/v) PLGA in DMF was applied to the surface of the plate. Then, the TCP film was immediately placed on the PLGA solution-applied surface of the plate. At the same time, the screw hole of the plate was placed in the rectangular frame hole of the TCP film. The plate having the TCP film placed thereon was freeze-dried in a vacuum for 24 hours.

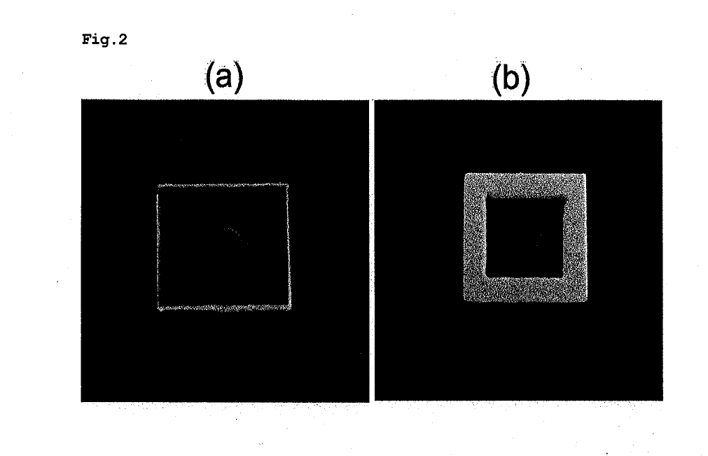

FIG. 2 shows optical images of a bone fixation plate before attachment of the TCP film (a) and a bone fixation plate after attachment of the TCP film (b).

### TEST EXAMPLE 1

### Examination of Physical and Chemical Properties of TCP Film

Scanning Electron Microscope (SEM) Analysis

For SEM imaging, the dried TCP film prepared in Example 2 was placed on an SEM sample mount and sputter-coated with platinum for 10 minutes (208HR, Cressington Scientific, England). Then, the sample was imaged with an SEM (7501F, Jeol, Japan).

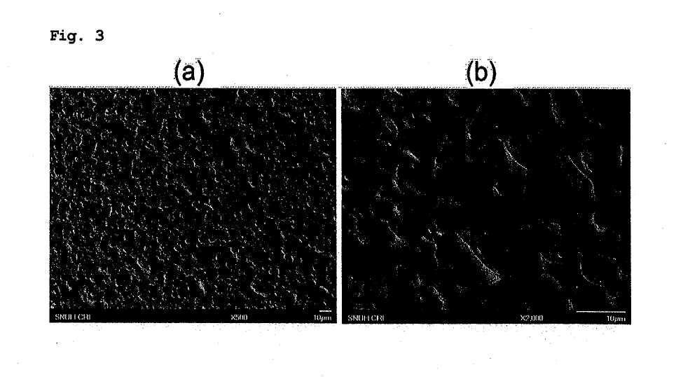

The results are shown in FIG. 3.

As can be seen in FIG. 3, the TCP particles on the TCP film were bound by PLGA, the TCP particles were dense.

X-ray Diffraction (XRD) Pattern

The sample TCP film was analyzed by scanning at 0.02° and 20=10-70° using an X-ray diffractometer (D/MAX RINT 2200-Ultima, Rigaku, Japan) equipped with Ni-filter Cu-Ka radiation (λ=1.5418 Å). β-TCP powder and PLGA powder were also examined for comparison with the TCP film sample and for the analysis of properties thereof.

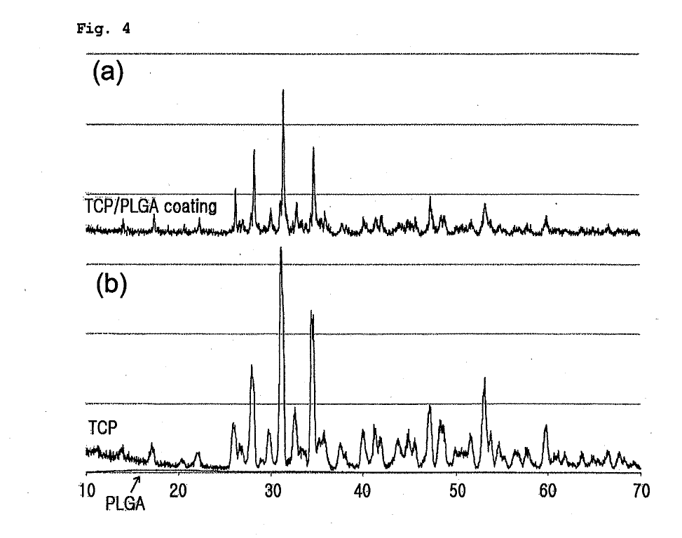

The results are shown in FIG. 4.

In FIG. 4, (a) shows the X-ray diffraction (XRD) pattern of β-TCP powder, and (b) shows the X-ray diffraction (XRD) pattern of the TCP film sample. In the PLGA powder, no X-diffraction pattern appeared.

As can be seen in FIG. 4, the TCP film sample showed a crystalline structure, like the β-TCP powder, whereas the PLGA powder had an amorphous structure.

### TEST EXAMPLE 2

### Analysis of X-ray Contrast Properties of TCP Film In Vitro

One-screw-hole samples of TCP film-attached plates having layer thicknesses of 0.5 mm and 1.3 mm were individually dipped in 10 mL of PBS (pH 7.4), and the bottom of the fixation plate and the bottom of a vial were fixed to each other by a double-sided adhesive tape and placed in a shaking incubator at 37° C. and 125 RPM. For 30 days, the sample was monitored visually and subjected to X-ray imaging (Manual mode, 55 kV, continous, HQ orthopedic mode) using a mobile C-arm (BV Pulsera, Philips, USA). The in vitro analysis was repeated three times.

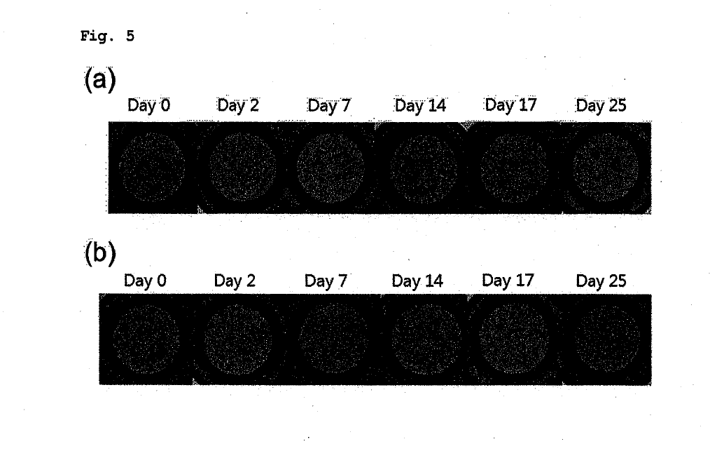

The results are shown in FIG. 5.

In FIG. 5, (a) shows the results of X-ray imaging of the TCP film-attached plate having a layer thickness of 1.3 mm with time, and (b) shows the results of X-ray imaging of the TCP film-attached plate having a layer thickness of 0.5 mm with time.

As can be seen in FIG. 5, X-ray visibility appeared in both the TCP film-attached plates having layer thicknesses of 0.5 mm and 1.3 mm for 25 days. Particularly, the TCP film-attached plate having a layer thickness of 1.3 mm showed a higher X-ray visibility.

Meanwhile, the X-ray images were monitored by densitometry using ImageJ software (National Institute of Health, USA), thereby quantifying the X-ray visibility. The measurement of densitometry was obtained from points throughout a visible TCP film square. The background sampled from an area near the visible square region was subtracted from the measurement of the TCP film to obtain the comparative value of the TCP film.

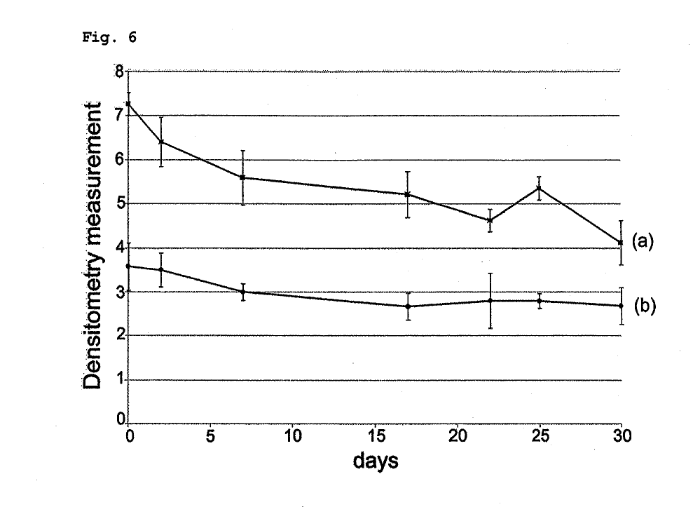

The results are shown in FIG. 6.

In FIG. 6, (a) shows the results of densitometry of the TCP film-attached plate having a layer thickness of 1.3 mm, and (a) shows the results of densitometry of the TCP film-attached plate having a layer thickness of 0.5 mm.

As can be seen in FIG. 6, X-ray visibility decreased with time in both the TCP film-attached plates having layer thicknesses of 1.3 mm and 0.5 mm. Particularly, the TCP film-attached plate having a layer thickness of 1.3 mm showed a higher X-ray visibility, not only at an initial time point, but also over all time points.

### TEST EXAMPLE 3

### Analysis of X-ray Contrast Properties of TCP Film In Vivo

X-ray Imaging with Time

Two rabbits (male New Zealand white) were used in in vivo analysis. In each rabbit, the one-screw-hole sample was surgically fixed to each of the humeri and to the right femur, and the control sample was fixed to the left femur. Thus, each rabbit had three samples and one control sample. Three 1.5 mm-thick thick samples were fixed to one rabbit, and three 0.5 mm-thick samples were fixed to the other rabbit.

The rabbits were monitored by X-ray imaging using a mobile C-arm (BV Pulsera, Philips, USA) for 3 months. The images were obtained by passing X-rays through the upper portions of the plates. Such images were analyzed by densitometry in the same manner as described in Example 2.

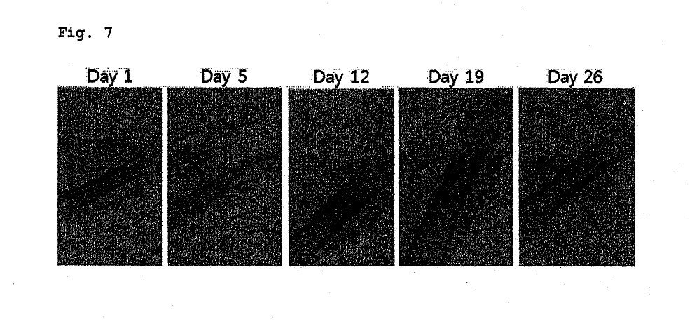

FIG. 7 shows X-ray images of the 1.5-mm-thick fixation plate with time.

As shown in FIG. 7, X-ray visibility decreases with time. This is because X-ray visibility decreases according to the degradation rate of the biodegradable polymer PLGA used in the present invention. In the case of the control plate to which no TCP film was attached, an X-ray image could not be obtained. However, as can be seen 24 hours after implantation of the TCP film-attached plate, an image could be obtained in the case of the TCP film-attached square plate having a hole, prepared in the present invention. X-ray measurements were carried out on days 5, 12, 19 and 26, and as a result, for up to 5 days the coated plate was clearly visible. At day 12, the image of the square became faint, and at day 26, the image of the plate completely disappeared.

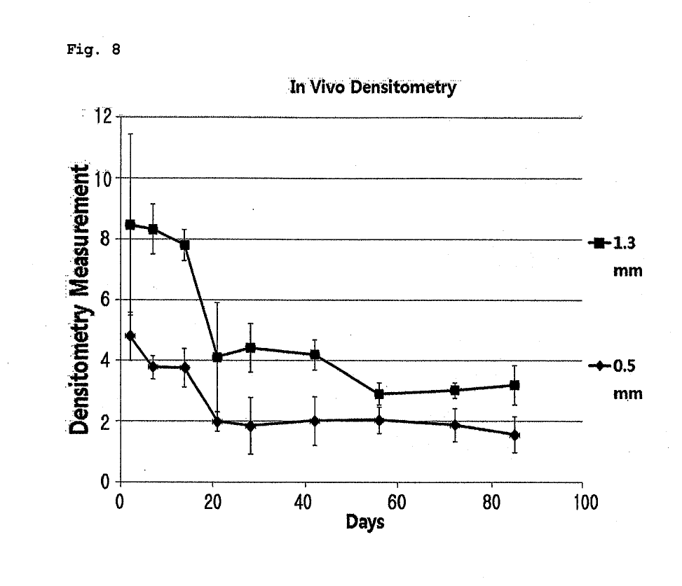

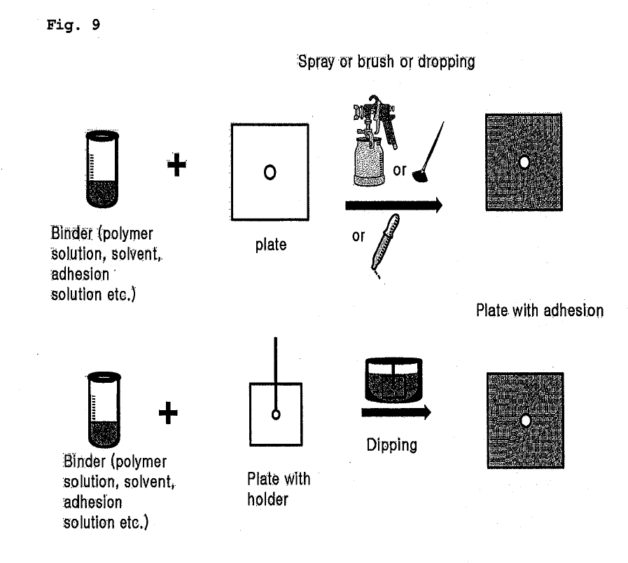

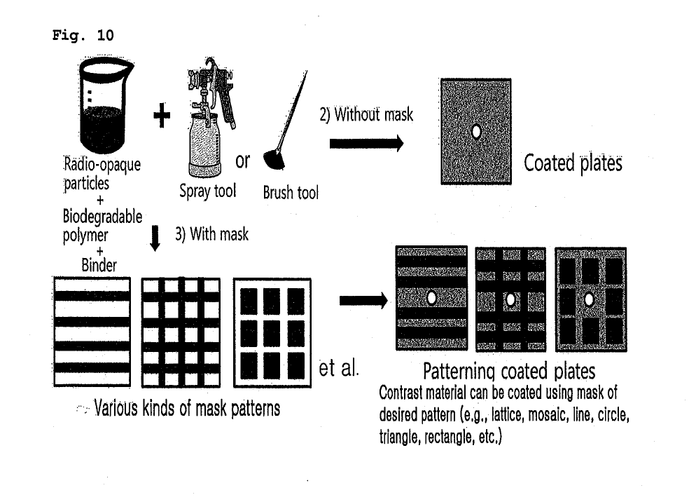

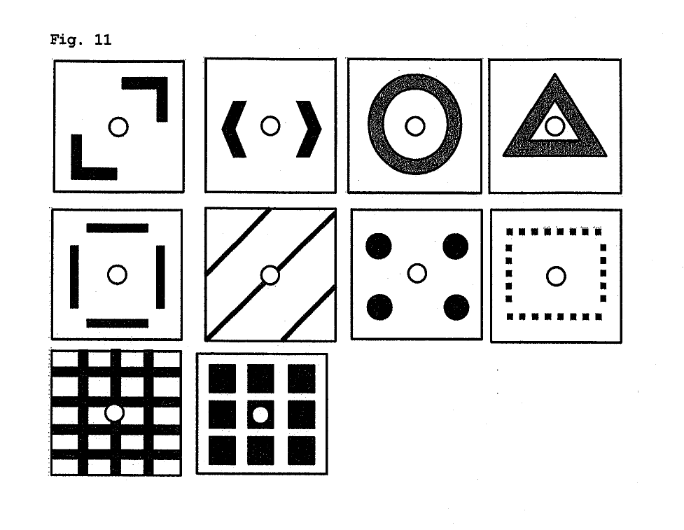

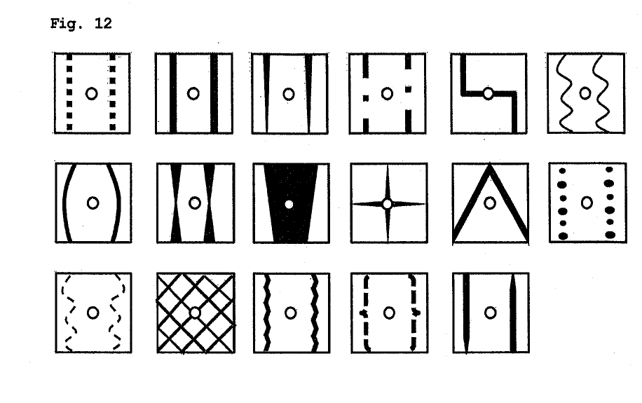

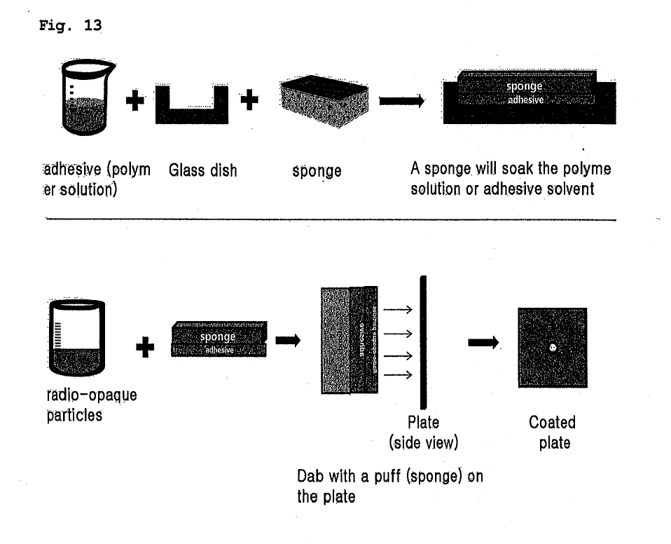

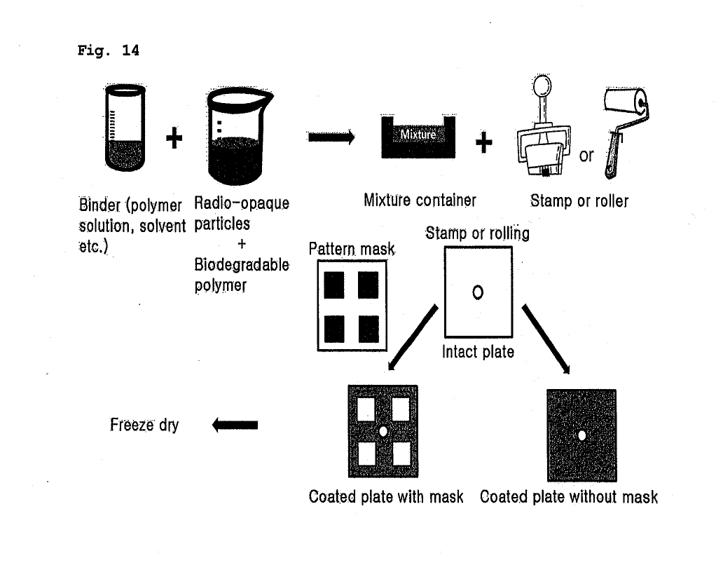

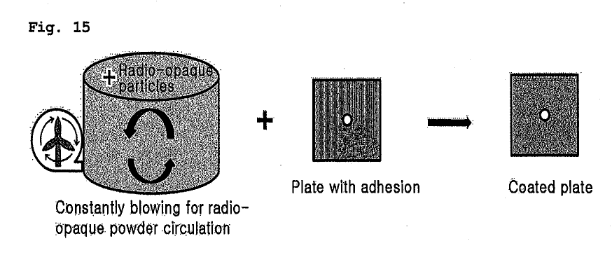

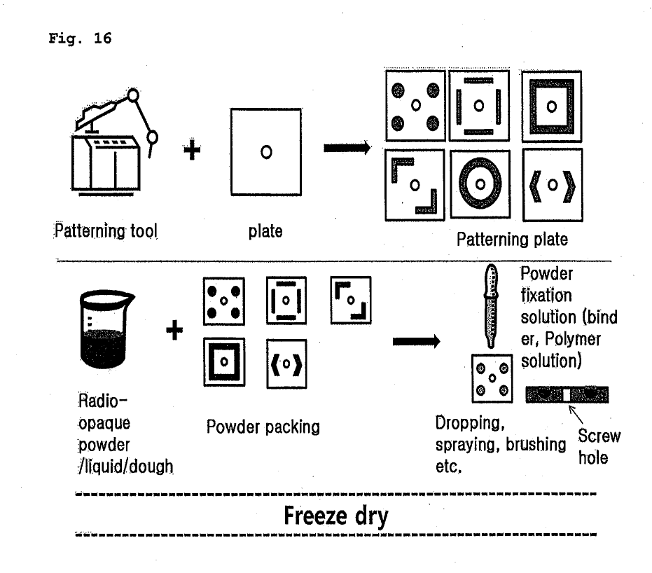

Meanwhile, FIG. 8 shows the results of densitometry that can explain a decrease in X-ray visibility with time.

FIG. 8 shows the results of analysis of the degradation rate of the TCP film-attached plates in rabbits. As can be seen in FIG. 8, the densitometry measurement decreased with time in both the plates having layer thicknesses of 1.3 mm and 0.5 mm. Further, at about 20 days, a rapid decrease in the densitometry measurement occurred in both the plates having different layer thicknesses, and this decrease appears to be attributable to the degradation rate of the biodegradable polymer PLGA used in the present invention. Thus, it can be concluded that the reason why the image of the plate cannot be seen in FIG. 8 after 20 days is because the densitometry measurement rapidly decreases after 20 days as can be seen from the results in FIG. 7.

What is claimed is:

1. An X-ray visible medical device comprising:
a medical device; and a layer bound onto the medical device, wherein the layer is composed of an X-ray contrast material or a composite of the X-ray contrast material and a biocompatible polymer.

2. The X-ray visible medical device of claim 1, wherein the medical device is any one selected from the group consisting of a plate, a screw and a pin, which is for bone fixation or bone fracture treatment.

3. The X-ray visible medical device of claim 1, wherein the biocompatible polymer is one or more selected from the group consisting of poly(lactic acid), poly(glycolic acid), poly(lactic-co-glycolic acid), poly(ethylene glycol), poly(trimethylene carbonate), poly(caprolactone), poly(dioxanone), poly(methylmethacrylate), polyethylene, polytetrafluoroethylene, polyvinyl chloride, polydimethylsiloxane, polyurethane, and copolymers thereof.

4. The X-ray visible medical device of claim 1, wherein the X-ray contrast material is one or more selected from the group consisting of calcium phosphate, barium sulfate, potassium iodide, metals, and combinations thereof.

5. The X-ray visible medical device of claim 1, wherein the layer is patterned.

6. The X-ray visible medical device of claim 5, wherein the shape of the pattern is a circle, a square, a triangle, a polygon, a straight line, a curved line, a dot, a letter, or a combination thereof.

7. The X-ray visible medical device of claim 1, wherein the layer is bound onto the medical device by an adhesive.

8. The X-ray visible medical device of claim 7, wherein the adhesive is one or more selected from among: one or more organic solvents selected from the group consisting of dimethylformamide (DMF), tetrahydrofuran (THF) and methylchloroform (MC); a polymer solution of one or more polymers selected from the group consisting of poly(methyl methacrylate), poly(lactic acid), poly(glycolic acid), poly(lactic-co-glycolic acid), poly(ethylene glycol), poly(trimethylene carbonate), and copolymers thereof, in the one or more organic solvents; and one or more biocompatible adhesives selected from the group consisting of fibrin glue, polysaccharides or mucopolysaccharide.

9. A method for preparing the X-ray visible medical device of claim 1, the method comprising the steps of:
(1) preparing a film composed of a composite of an X-ray contrast material and a biocompatible polymer; and
(2) binding the film onto a medical device.

10. The method of claim 9, wherein step (1) comprises the steps of:
(1-1) mixing a biocompatible polymer solution or melt with an X-ray contrast material;
(1-2) placing the mixture in a mold to prepare a film; and
(1-3) drying the film.

11. The method of claim 9, wherein step (2) comprises the steps of:
(2-1) coating the medical device with an adhesive;
(2-2) placing the film composed of the composite of the X-ray contrast material and the biocompatible polymer on the adhesive-coated medical device; and
(2-3) drying the medical device having the film placed thereon.

12. The method of claim 9, wherein the shape of the film is a circular, square, triangular or polygonal shape.

13. A method for preparing the X-ray visible medical device of claim 1, the method comprising the steps of:
(1) preparing an X-ray contrast material or a mixture of the X-ray contrast material and a biocompatible polymer;
(2) coating a medical device with an adhesive; and
(3) binding the X-ray contrast material or mixture of step (1) onto the adhesive-coated medical device of step (2) to form a layer on the medical device.

14. The method of claim 13, wherein the method further comprises, step (4) of drying the medical device having the layer bound thereto.

15. The method of claim 13, wherein the binding in step (3) is carried out by spreading, spraying, dropping, dipping, coating, dabbing with sponge, stamping, rolling, brushing, spray casting or electrospinning.

16. The method of claim 13, wherein the binding in step (3) is carried out using a mask to pattern the layer.

17. The method of claim 16, wherein the shape of the pattern is a circle, a square, a triangle, a polygon, a straight line, a curved line, a dot, a letter, or a combination thereof.

18. A method for preparing the X-ray visible medical device of claim 1, the method comprising the steps of:
(1) preparing a mixture of an X-ray contrast material and an adhesive or a mixture of the X-ray contrast material, a biocompatible polymer and the adhesive; and
(2) binding the mixture onto a medical device to form a layer on the medical device.

19. The method of claim 18, wherein the method further comprises, step (3) of drying the medical device having the layer bound thereto.

20. The method of claim 18, wherein the binding in step (2) is carried out by spreading, spraying, dropping, dipping, coating, dabbing with sponge, stamping, rolling, brushing, spray casting or electrospinning.

21. The method of claim 18, wherein the binding in step (2) is carried out using a mask to pattern the layer.

22. The method of claim 21, wherein the shape of the pattern is a circle, a square, a triangle, a polygon, a straight line, a curved line, a dot, a letter, or a combination thereof.

23. A method for preparing the X-ray visible medical device of claim 1, the method comprising the steps of:
(1) forming a groove on a medical device;
(2) preparing an X-ray contrast material or a mixture of the X-ray contrast material and a biocompatible polymer;
(3) packing the X-ray contrast material or the X-ray contrast material/biocompatible polymer of step (2) into the groove formed on the medical device in step (1); and
(4) applying an adhesive to the packed groove.

24. The method of claim 23, wherein the method further comprises, step (5) drying the adhesive-applied medical device.

25. The method of claim 23, wherein the groove formed in step (1) is patterned.

26. The method of claim 25, wherein the shape of the pattern is a circle, a square, a triangle, a polygon, a straight line, a curved line, a dot, a letter, or a combination thereof.
